(19) United States
(12) Patent Application Publication
(10) Pub. No.: US 2006/0013853 A1
(43) Pub. Date: Jan. 19, 2006

(54) Medical devices having conductive substrate and covalently bonded coating layer

(76) Inventor: Robert E. Richard, Wrentham, MA (US)

Correspondence Address: MAYER, FORTKORT & WILLIAMS, PC, 251 NORTH AVENUE WEST, 2ND FLOOR, WESTFIELD, NJ, 07090, US

(21) Appl. No.: 10/894,391

(22) Filed: Jul. 19, 2004

(51) Int. Cl. A61F 2/00

(52) U.S. Cl. 424/423

(57) ABSTRACT

The present invention provides a medical device comprising an electrically conductive substrate and a coating layer that covers at least a portion of the electrically conductive substrate, wherein the coating layer comprises a polymer that is made by a process that comprises (a) electrochemically linking a initiator to a surface of the electrically conductive substrate and (b) conducting a free radical polymerization reaction in the presence of one or more free radical polymerizable monomers.

### FIELD OF THE INVENTION

The present invention relates generally to medical devices which contain polymeric surface coatings. The present invention also relates to methods for producing covalently bonded polymeric coatings for medical devices, particularly for insertable or implantable medical devices.

### BACKGROUND OF THE INVENTION

Numerous polymer-based medical devices have been developed for the delivery of therapeutic agents to the body. In accordance with some typical delivery strategies, a therapeutic agent is provided within a polymeric carrier layer and/or beneath a polymeric barrier layer that is associated with a medical device. Once the medical device is placed at the desired location within a patient, the therapeutic agent is released from the medical device at a rate that is dependent upon the nature of the polymeric carrier and/or barrier layer.

Materials which are suitable for use in making implantable or insertable medical devices typically exhibit one or more of the qualities of exceptional biocompatibility, extrudability, elasticity, moldability, good fiber forming properties, tensile strength, durability, and the like. Moreover, the physical and chemical characteristics of the device materials can play an important role in determining the final release rate of the therapeutic agent.

As a specific example, block copolymers of polyisobutylene and polystyrene, for example, polystyrene-polyisobutylene-polystyrene triblock copolymers (SIBS copolymers), which are described in U.S. Pat. No. 6,545,097 to Pinchuk et al., hereby incorporated by reference in its entirety, have proven valuable as release polymers in implantable or insertable drug-releasing medical devices. As described in Pinchuk et al., the release profile characteristics of therapeutic agents such as paclitaxel from SIBS copolymer systems demonstrate that these copolymers are effective drug delivery systems for providing therapeutic agents to sites in vivo. These copolymers are particularly useful for medical device applications because of their excellent strength, biostability and biocompatibility, particularly within the vasculature. For example, SIBS copolymers exhibit high tensile strength, which frequently ranges from 2,000 to 4,000 psi or more, and resist cracking and other forms of degradation under typical in vivo conditions. Biocompatibility, including vascular compatibility, of these materials has been demonstrated by their tendency to provoke minimal adverse tissue reactions (e.g., as measured by reduced macrophage activity). In addition, these polymers are generally hemocompatible as demonstrated by their ability to minimize thrombotic occlusion of small vessels when applied as a coating on coronary stents.

Currently, technologies used to prepare coatings on medical devices having metal surfaces, which are typical of coronary stents and many other medical devices, include spray and dip coating. While these processes are capable of producing effective coatings, there is a need for a more efficient and precise coating method. One major drawback of the coatings produced using these prior art methods is that they may lack mechanical integrity since they envelop the device, but do not necessarily chemically adhere to it. Another potential drawback is that spray coatings and dip coatings are physical, rather than chemical, coatings. Because they rely upon the spray or dip equipment and not upon chemical interactions at a molecular level for controlling the coating process, precision in terms of thickness and conformity with the substrate surface has been difficult to achieve with these techniques.

Attempts have been made to address the poor adhesion problem, including first coating the metal surface with one or more “primer layers,” followed by application of the polymeric coating of interest. One obvious disadvantage of such a primer is that it introduces another layer which does not aid, and may even hinder, the drug delivery functions of the polymeric coating. In addition, the polymers or materials suitable for use as a primer layer may not necessarily possess the desired mechanical or biocompatible characteristics, and thus the range of materials that may be utilized as part of a coating system for a medical device may be limited.

In view of the above, it would be advantageous to provide coatings that have desirable biostability and/or biocompatibility properties, while also having improved mechanical properties. It would also be desirable to provide tenacious coatings which exhibit improved adhesion to the surfaces of common medical device substrates.

### SUMMARY OF THE INVENTION

These and other challenges of the prior art are addressed by the present invention which provides a medical device comprising an electrically conductive substrate and a coating layer that covers at least a portion of the electrically conductive substrate. The coating layer comprises a polymer, which is made by a process that comprises (a) electrochemically linking a free radical polymerization initiator to a surface of the electrically conductive substrate and (b) conducting a free radical polymerization reaction in the presence of one or more free radical polymerizable monomers.

The present invention is advantageous in that coated medical devices are provided in which the coating is covalently bonded to the surface of the medical device, thereby providing a strong, conformal coating.

Another advantage of the present invention is that implantable or insertable medical devices are provided, which result in controlled release of a therapeutic agent.

Yet another advantage of the present invention is that methods of coating medical devices are provided which avoid various limitations of standard dip and spray coating processes.

These and other embodiments and advantages of the present invention will become immediately apparent to those of ordinary skill in the art upon review of the Detailed Description and claims to follow.

### BRIEF DESCRIPTION OF THE DRAWINGS

FIG. 1 schematically illustrates a two-step process for the grafting of polystyrene to an electrically conducting substrate.

FIG. 2 is an SEM image of polystyrene grafted to a steel substrate surface.

### DETAILED DESCRIPTION OF THE INVENTION

The present invention relates to medical devices having polymeric coating layers wherein covalent bonds exist at the interfaces between the coating layers and the substrates on which they are formed, and to methods for producing such coating layers. Specifically, the present invention relates to processes for chemically linking (e.g., covalently bonding) a polymer to a substrate such as an electrically conductive substrate (e.g., a metal surface) by electrochemically attaching an initiator species to the substrate followed by a monomer polymerization reaction such as atom-transfer radical polymerization (ATRP). The processes of the present invention can be used for a number of purposes, including the preparation of passive device coatings, hydrophilic device coatings, and device coatings that can be loaded with one or more therapeutic agents for in situ delivery of the agent after implantation within a patient.

For purposes of clarity, as used herein, polymers are molecules containing one or more chains, which contain multiple copies of one or more constitutional units. An example of a common polymer is polystyrene 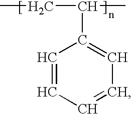
where n is an integer, typically an integer of 10 or more, more typically on the order of 10's, 100's, 1000's or even more, in which the constitutional units in the chain correspond to styrene monomers: 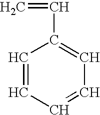
(i.e., they originate from, or have the appearance of originating from, the polymerization of styrene monomers in this case, the addition polymerization of styrene monomers). As used herein, copolymers are polymers that contain at least two dissimilar constitutional units.

As used herein, a polymer “block” is a grouping of 10 or more constitutional units, commonly 20 or more, 50 or more, 100 or more, 200 or more, 500 or more, or even 1000 or more units. A “chain” is a linear (unbranched) grouping of 10 or more constitutional units (i.e., a linear block).

In one aspect, the invention provides a medical device that comprises a substrate, preferably an electrically conductive substrate, and a coating layer that covers at least a portion of the substrate. The coating layer contains one or more polymers and is made by a process that includes (a) electrochemically linking an initiator to a surface of the electrically conductive substrate and (b) conducting a free radical polymerization reaction in the presence of a free radical polymerizable monomer, or two or more free radical polymerizable comonomers.

The technology for linking the initiator to the electrically conductive surface, for example, a stainless steel surface, includes electrografting processes such as those disclosed by Claes et al., Polymer Coating of Steel by a Combination of Electrografting and Atom-Transfer Radical Polymerization, Macromolecules, Web release No. 0217130, published Jul. 19, 2003, the contents of which are hereby incorporated by reference in their entirety.

The usually weak and short-term adhesion between organic polymers and radically different materials such as metals, glass, and carbon have presented special challenges in formulating suitable coatings. For example, proper adhesion of the polymer to a medical device having a metallic surface (e.g., a coronary stent) can often be one of the most important factors in the successful implementation of a medical device.

The electrografting method overcomes this and other challenges by forming a tenacious chemical link between a functional group of an initiator and the metal atoms of a substrate such as stainless steel. The electrografting processes comprises applying an electric potential to the electrically conductive substrate in the presence of the initiator, with the ensuing reaction, for example, a reduction reaction, creating a link between the initiator and the substrate. Such a link establishes a covalent bond at the substrate surface such that a strong adhesion is established between the resulting polymeric coating and the substrate surface. The final polymer coating may be formed by polymerization according to various known polymerization methods, including atom-transfer radical polymerization (ATRP), among others.

Examples of some suitable substrates for the practice of the present invention include but is not limited to, electrically conductive substrates comprising an elemental transition metal or alloy, including metals such as copper, nickel, tantalum, silver, gold, platinum, palladium, iridium, osmium, rhodium, titanium, tungsten, ruthenium and metal alloys such as iron-chromium alloys (e.g., stainless steel, which typically contains at least 50% iron and at least 11.5% chromium), nickel-titanium alloys, nickel-chromium alloys (e.g., INCONEL® alloys), cobalt chromium alloys, platinum-enriched stainless steel or combinations of two or more metals or metal alloys.

Not all of the electrically conductive substrate needs to be conductive, but rather only those portions to which it is desired to attach the initiator. Hence, the conductive substrate can be, for example, a solid metal substrate, a non-conductive substrate having a metallic coating, and so forth.

In general, the initiator will have at least one functionality that is conducive to electrografting and at least one functionality that is able to initiate free radical polymerization (e.g., an activated halide functionality, which is able to initiate ATRP polymerization of, for example, vinyl monomers). One specific example is 2-chloropropionate ethyl acrylate (cPEA).

In some embodiments, the initiator is a polymeric macro-initiator, e.g., a polymeric macro-initiator containing at least one functionality that is conducive to electrografting and at least one further functionality that is able to initiate free radical polymerization such as ATRP. In some embodiments, the initiator comprises an electrochemically linkable group comprising any alkyl halide with one or more activating groups on the α carbon (such as aryl, carbonyl, allyl, and the like). Also, the initiator may comprise a polyhalogenated compound or compounds with a weak R—X bond such as N—X, S—X, or O—X, where X is a halogen atom, such as fluorine, chlorine, bromine, iodine, etc. Examples include polymers of cPEA, copolymers of cPEA (e.g., poly[cPEA-co-ethyl acrylate]), cPEA-terminated poly(alkylacrylates), and other polymers containing an activated-halide functionality, which are capable of being electrochemically grafted to a conductive substrate.

In some embodiments, a difunctional free radical initiator such as dimethyl-2,6-heptanedioate is used to initiate polymerization (e.g., of a alkyl acrylate monomer such as ethyl acrylate), thereby forming a polyacrylate macro-initiator having a functionality (e.g., an acrylate functionality) that can form a covalent bond with a conductive substrate surface.

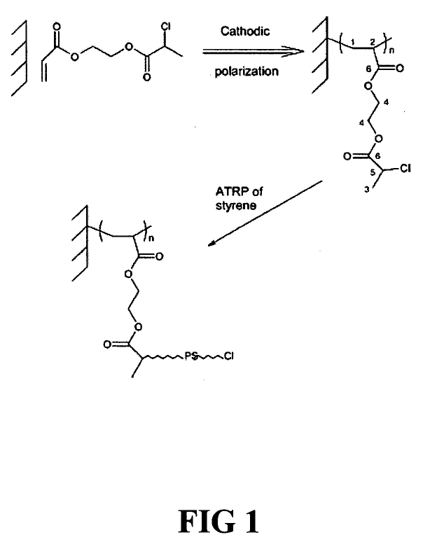

As previously noted, the initiator is chemically linked to an electrochemically conductive substrate surface (e.g., a stainless steel surface) by electrografting the initiator to the substrate surface, thereby forming a covalent bond between the substrate surface and the initiator. For example, in certain preferred embodiments, the initiator is a poly(2-chloropropionate ethyl acrylate) macro-initiator, which is synthesized using known methods. In addition to bearing an acrylate functional group that is amenable to electrografting, the molecule also possesses an activated chloride that is able to initiate the controlled radical polymerization of monomers such as vinyl monomers by ATRP. An exemplary scheme for the grafting of polystyrene onto an electrically conductive substrate, and as published in Claes et al., cited above, is illustrated in FIG. 1. Upon application of an electric potential, electroreduction of the acrylate occurs at the electrically conductive surface, which serves in this instance as a cathode, leading to the rapid formation of a film on the same. Poly(2-chloropropionate ethyl acrylate), electrografted on steel which is a non-noble metal, forms a strongly adhering macro-initiator for the ATRP of a monomer such as styrene or other vinyl monomer.

Once electrochemically attached, initiators such as those described above can be used to synthesize a variety of polymers according to various known methods, including ionic and various radical polymerization methods such as azobis(isobutyronitrile)- or peroxide-initiated processes, controlled/“living” radical polymerizations such as atom transfer radical polymerization (ATRP), stable free-radical polymerization (SFRP), nitroxide-mediated processes (NMP), and degenerative transfer (e.g., reversible addition-fragmentation chain transfer (RAFT)) processes.

In particular, ATRP is a preferred, versatile process by which the chemical architecture of a polymer can be controlled very closely, and which process can be used with a wide variety of monomers to create polymers having a diverse range of chemical characteristics (hydrophobic, hydrophilic, ionic, etc.). Because ATRP is tolerant of a variety of functional groups (e.g., alcohol, amine, carboxylic, acid, sulfonate), a combination of electrografting with subsequent ATRP is preferred in many embodiments. ATRP and the other polymerization methods described herein are well-detailed in the literature and are described, for example, in an article by Pyun and Matyjaszewski, “Synthesis of Nanocomposite Organic/Inorganic Hybrid Materials Using Controlled/“Living” Radical Polymerization,” Chem. Mater., 13:3436-3448 (2001), the contents of which are incorporated by reference in their entirety.

In polymerizations of a monomer (M, in scheme below) via ATRP, radicals are generated by the redox reaction of organic halides such as alkyl halides (RX, in scheme below) with transition-metal complexes (Met⁺ⁿ, in scheme below). Radicals can then propagate, but are rapidly deactivated by the oxidized form of the transition-metal catalyst. The initiators typically used are haloesters (e.g., 1-butyl chloropropionate, 2-chloropropionate ethyl acrylate, ethyl 2-boroisobutyrate and methyl 2-bromopropionate), or benzyl halides (e.g., 1-phenylethyl bromide and benzyl bromide). A wide range of transition-metal complexes, such as Ru- (e.g., Grubbs catalyst), Cu-, and Fe-based systems are employed in ATRP. For Cu-based systems, ligands such as 2,2′-bipyridine and aliphatic amines are typically employed to control both the solubility and activity of various ATRP catalysts. A typical ATRP mechanism is illustrated by the following scheme:

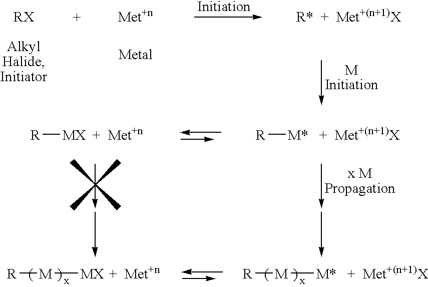

Using these and other techniques described herein, a variety of polymers may be chemically bonded to conductive medical device surfaces, depending on the goal to be achieved. For example, a polymer may be chosen to impart specific properties of the medical device, e.g., to render the surface more hydrophilic or hydrophobic, to render the surface adhesive or reduce friction during implantation or delivery, to render the surface biocompatible or passive, to make the surface more resistant to environmental or biological attack, to release a therapeutic agent from the surface, and so forth.

The polymers that may be employed include homopolymers or copolymers (such as alternating, random, statistical, tapered/gradient and block copolymers), may be cyclic, linear or branched (e.g., the polymers may have star, comb or dendritic architecture), they may be natural or synthetic, or they may be thermoplastic or thermosetting.

In embodiments where it is desirable to synthesize polymer blocks having a main chain and a plurality of side chains, the polymerization can proceed, for example, in the presence of (i) a macro-monomer, which comprises the side chain and has a free-radical polymerizable end group and (ii) optionally, a free-radical polymerizable comonomer or a combination of free-radical polymerizable comonomers. Preferably, the end group of the macro-monomer is terminally unsaturated and the polymerizable comonomer is an unsaturated monomer.

A “macro-monomer” as the term is used herein is a macromolecule, commonly a polymer, which has a reactive group, often an end-group, which enables it to act as a monomer molecule, contributing a single monomeric unit to a chain of the final macromolecule. For example, a long-chain vinyl polymer or vinyl oligomer (as used herein, an oligomer is a polymer containing from 2-9 constitutional units) that has a polymerizable double bond at the end of the chain is a macromonomer. Homopolymerization or copolymerization of a macromonomer yields comb or graft polymers.

Examples of monomers that can be used in the polymerization reactions of the present invention include unsaturated monomers such as alkyl methacrylates, alkyl acrylates, hydroxyalkyl methacrylates, vinyl esters, vinyl aromatics such as styrene, α-methylstyrene, macro-monomers having free radical polymerizable end groups, and combinations thereof. Examples of macro-monomers include those having a polysiloxane block, a polyisobutylene block, a poly(vinyl aromatic) block such as a polystyrene block, a polyethylene oxide block, a polyvinylpyrrolidone block, a polymethylmethacrylate block, or a combination thereof, and having a free radical polymerizable end group.

In some specific embodiments, the polymer formed comprises a low T_{g} polymer block or a high T_{g} polymer block. In other specific embodiments, the polymer formed is a copolymer that comprises (i) a low T_{g} polymer block and (ii) a high T_{g} polymer block, for example, wherein the macro-initiator comprises a free radical terminated low T_{g} polymer block (or a free radical terminated high T_{g} polymer block). In still other specific embodiments, the polymer is a copolymer that further comprises a low T_{g} block and a graft copolymer block comprising a main chain and a plurality of side chains, wherein the macro-initiator comprises a free radical terminated low T_{g} polymer block and wherein the radical polymerization reaction is conducted in the presence of a macro-monomer comprising the side chain and a free radical polymerizable end group.

A “low T_{g} polymer block” is a polymer block that displays one or more glass transition temperatures (T_{g}), as measured by any of a number of techniques including differential scanning calorimetry (DSC), dynamic mechanical analysis (DMA), or dielectric analysis (DEA), that is below ambient temperature, more typically below 25° C., below 0° C., below −25° C., or even below −50° C. “Ambient temperature” is typically 25° C.-45° C., more typically body temperature (e.g., 35° C.-40° C.). As a result of their low glass transition temperature, low T_{g} polymer blocks are typically elastomeric at ambient temperature. Homopolymers of some low T_{g} polymer blocks, such as linear or branched silicone (e.g. polydimethylsiloxane), are viscous liquids or millable gums at room temperature and become elastomeric upon covalent cross-linking.

Conversely, an elevated or “high T_{g} polymer block” is a polymer block that displays one or more glass transition temperatures, as measured by any of a number of techniques including differential scanning calorimetry, dynamic mechanical analysis, or thermomechanical analysis, which is above ambient temperature, more typically above 50° C., above 60° C., above 70° C., above 80° C., above 90° C. or even above 100° C.

Hence, copolymers having one or more low T_{g} blocks and one or more high T_{g} polymer blocks will typically have one or more glass transition temperatures below ambient temperature and one or more glass transition temperatures above ambient temperature. This typically results in the formation of rubbery and hard phases within the coating layer at ambient temperatures.

The low and high T_{g} polymer blocks may be present in the copolymers, for example, as interior blocks or as endblocks. The low and high T_{g} polymer blocks may be provided in a variety of configurations, including cyclic, linear and branched configurations. Branched configurations include star-shaped configurations (e.g., configurations in which three or more chains emanate from a single branch point), comb configurations (e.g., configurations having a main chain and a plurality of branching side chains) and dendritic configurations (e.g., arborescent and hyperbranched polymers). The low and high T_{g} polymer blocks may contain, for example, a repeating series of units of a single type, a series of units of two or more types in a repeating (e.g., alternating), random, statistical or gradient distribution, and so forth.

Specific examples of low T_{g} polymer blocks from which the low T_{g} polymer blocks of the present invention can be selected include homopolymer blocks and copolymer blocks formed from (or having the appearance of being formed from) one or more of the following: acrylic monomers, methacrylic monomers, vinyl ether monomers, cyclic ether monomers, ester monomers, unsaturated hydrocarbon monomers, including alkene monomers, halogenated alkene monomers, halogenated unsaturated hydrocarbon monomers, and siloxane monomers. Numerous specific examples are listed below. The T_{g} values are published values for homopolymers of the listed monomeric unit.

Specific acrylic monomers include: (a) alkyl acrylates such as methyl acrylate (T_{g} 10° C.), ethyl acrylate (T_{g} −24° C.), propyl acrylate, isopropyl acrylate (T_{g} −11° C., isotactic), butyl acrylate (T_{g} −54° C.), sec-butyl acrylate (T_{g} −26° C.), isobutyl acrylate (T_{g} −24° C.), cyclohexyl acrylate (T_{g} 19° C.), 2-ethylhexyl acrylate (T_{g} −50° C.), dodecyl acrylate (T_{g} −3° C.) and hexadecyl acrylate (T_{g} 35° C.), (b) arylalkyl acrylates such as benzyl acrylate (T_{g} 6° C.), (c) alkoxyalkyl acrylates such as 2-ethoxyethyl acrylate (T_{g} −50° C.) and 2-methoxyethyl acrylate (T_{g} −50° C.), (d) halo-alkyl acrylates such as 2,2,2-trifluoroethyl acrylate (T_{g} −10° C.) and (e) cyano-alkyl acrylates such as 2-cyanoethyl acrylate (T_{g} 4° C.).

Specific methacrylic monomers include (a) alkyl methacrylates such as butyl methacrylate (T_{g} 20° C.), hexyl methacrylate (T_{g} −5° C.), 2-ethylhexyl methacrylate (T_{g} −10° C.), octyl methacrylate (T_{g} −20° C.), dodecyl methacrylate (T_{g} −65° C.), hexadecyl methacrylate (T_{g} 15° C.) and octadecyl methacrylate (T_{g} −100° C.) and (b) aminoalkyl methacrylates such as diethylaminoethyl methacrylate (T_{g} 20° C.) and 2-tert-butyl-aminoethyl methacrylate (T_{g} 33° C.).

Specific vinyl ether monomers include (a) alkyl vinyl ethers such as methyl vinyl ether (T_{g} −31° C.), ethyl vinyl ether (T_{g} −43° C.), propyl vinyl ether (T_{g} −49° C.), butyl vinyl ether (T_{g} −55° C.), isobutyl vinyl ether (T_{g} −19° C.), 2-ethylhexyl vinyl ether (T_{g} −66° C.) and dodecyl vinyl ether (T_{g} −62° C.).

Specific cyclic ether monomers include tetrahydrofuran (T_{g} −84° C.), trimethylene oxide (T_{g} −78° C.), ethylene oxide (T_{g} −66° C.), propylene oxide (T_{g} −75° C.), methyl glycidyl ether (T_{g} −62° C.), butyl glycidyl ether (T_{g} −79° C.), allyl glycidyl ether (T_{g} −78° C.), epibromohydrin (T_{g} −14° C.), epichlorohydrin (T_{g} −22° C.), 1,2-epoxybutane (T_{g} −70° C.), 1,2-epoxyoctane (T_{g} −67° C.) and 1,2-epoxydecane (T_{g} −70° C.).

Specific ester monomers (other than acrylates and methacrylates) include ethylene malonate (T_{g} −29° C.), vinyl acetate (T_{g} 30° C.), and vinyl propionate (T_{g} 10° C.).

Specific alkene monomers include ethylene, propylene (T_{g} −8 to −13° C.), isobutylene (T_{g} −73° C.), 1-butene (T_{g} −24° C.), trans-butadiene (T_{g} −58° C.), 4-methyl pentene (T_{g} 29° C.), 1-octene (T_{g} −63° C.) and other α-olefins, cis-isoprene (T_{g} −63° C.), and trans-isoprene (T_{g} −66° C.).

Specific halogenated alkene monomers include vinylidene chloride (T_{g} −1 8° C.), vinylidene fluoride (T_{g} −40° C.), cis-chlorobutadiene (T_{g} −20° C.), and trans-chlorobutadiene (T_{g} −40° C.).

Specific siloxane monomers include dimethylsiloxane (T_{g} −127° C.), diethylsiloxane, methylethylsiloxane, methylphenylsiloxane (T_{g} −86° C.), and diphenylsiloxane.

Specific examples of high T_{g} polymer blocks include homopolymer blocks and copolymer blocks formed from (or having the appearance of being formed from) one or more of the following: various vinyl aromatic monomers, other vinyl monomers, other aromatic monomers, methacrylic monomers, and acrylic monomers. Numerous specific examples are listed below. The T_{g} values are published values for homopolymers of the listed monomeric unit.

Vinyl aromatic monomers are monomers having aromatic and vinyl moieties, including unsubstituted monomers, vinyl-substituted monomers and ring-substituted monomers. Several specific vinyl aromatic monomers follow: (a) unsubstituted vinyl aromatics, such as atactic styrene (T_{g} 100° C.), isotactic styrene (T_{g} 100° C.) and 2-vinyl naphthalene (T_{g} 151° C.), (b) vinyl substituted aromatics such as methyl styrene, (c) ring-substituted vinyl aromatics including (i) ring-alkylated vinyl aromatics such as 3-methylstyrene (T_{g} 97° C.), 4-methylstyrene (T_{g} 97° C.), 2,4-dimethylstyrene (T_{g} 112° C.), 2,5-dimethylstyrene (T_{g} 143° C.), 3,5-dimethylstyrene (T_{g} 104° C.), 2,4,6-trimethylstyrene (T_{g} 162° C.), and 4-tert-butylstyrene (T_{g} 127° C.), (ii) ring-alkoxylated vinyl aromatics, such as 4-methoxystyrene (T_{g} 113° C.) and 4-ethoxystyrene (T_{g} 86° C.), (iii) ring-halogenated vinyl aromatics such as 2-chlorostyrene (T_{g} 119° C.), 3-chlorostyrene (T_{g} 90° C.), 4-chlorostyrene (T_{g} 110° C.), 2,6-dichlorostyrene (T_{g} 167° C.), 4-bromostyrene (T_{g} 118° C.) and 4-fluorostyrene (T_{g} 95° C.) and (iv) ester-substituted vinyl aromatics such as 4-acetoxystyrene (T_{g} 116° C.).

Other specific vinyl monomers include: (a) vinyl alcohol (T_{g} 85° C.); (b) vinyl esters such as vinyl benzoate (T_{g} 71° C.), vinyl 4-tert-butyl benzoate (T_{g} 101° C.), vinyl cyclohexanoate (T_{g} 76° C.), vinyl pivalate (T_{g} 86° C.), vinyl trifluoroacetate (T_{g} 46° C.), vinyl butyral (T_{g} 49° C.), (c) vinyl amines such as 2-vinyl pyridine (T_{g} 104° C.), 4-vinyl pyridine (T_{g} 142° C.), and vinyl carbazole (T_{g} 227° C.), (d) vinyl halides such as vinyl chloride (T_{g} 81° C.) and vinyl fluoride (T_{g} 40° C.); (e) alkyl vinyl ethers such as tert-butyl vinyl ether (T_{g} 88° C.) and cyclohexyl vinyl ether (T_{g} 81° C.), and (f) other vinyl compounds such as 1-vinyl-2-pyrrolidone (T_{g} 54° C.) and vinyl ferrocene (T_{g} 189° C.).

Specific aromatic monomers, other than vinyl aromatics, include: acenaphthalene (T_{g} 214° C.) and indene (T_{g} 85° C.).

Specific methacrylic monomers include (a) methacrylic acid (T_{g} 228° C.), (b) methacrylic acid salts such as sodium methacrylate (T_{g} 310° C.), (c) methacrylic acid anhydride (T_{g} 159° C.), (d) methacrylic acid esters (methacrylates) including (i) alkyl methacrylates such as atactic methyl methacrylate (T_{g} 105-120° C.), syndiotactic methyl methacrylate (T_{g} 115° C.), ethyl methacrylate (T_{g} 65° C.), isopropyl methacrylate (T_{g} 81° C.), isobutyl methacrylate (T_{g} 53° C.), t-butyl methacrylate (T_{g} 118° C.) and cyclohexyl methacrylate (T_{g} 92° C.), (ii) aromatic methacrylates such as phenyl methacrylate (T_{g} 110° C.) and including aromatic alkyl methacrylates such as benzyl methacrylate (T_{g} 54° C.), (iii) hydroxyalkyl methacrylates such as 2-hydroxyethyl methacrylate (T_{g} 57° C.) and 2-hydroxypropyl methacrylate (T_{g} 76° C.), (iv) additional methacrylates including isobornyl methacrylate (T_{g} 110° C.) and trimethylsilyl methacrylate (T_{g} 68° C.), and (e) other methacrylic-acid derivatives including methacrylonitrile (T_{g} 120° C.).

Specific acrylic monomers include (a) acrylic acid (T_{g} 105° C.), its anhydride and salt forms, such as potassium acrylate (T_{g} 194° C.) and sodium acrylate (T_{g} 230° C.); (b) certain acrylic acid esters such as tert-butyl acrylate (T_{g} 43-107° C.) (Tₘ 193° C.), hexyl acrylate (T_{g} 57° C.) and isobornyl acrylate (T_{g} 94° C.); (c) acrylic acid amides such as acrylamide (T_{g} 165° C.), N-isopropylacrylamide (T_{g} 85-130° C.) and N,N dimethylacrylamide (T_{g} 89° C.); and (d) other acrylic-acid derivatives including acrylonitrile (T_{g} 125° C.).

In some embodiments, the coating layers of the present invention are loaded with a therapeutic agent. For example, once an adherent coating has been established via electrografting and ATRP or another polymerization technique, solvent-based methods may be utilized to introduce a therapeutic agent into the polymer coating.

Where solvent-based techniques are used to introduce a therapeutic agent into the coating layer, the solvent system that is selected will contain one or more solvent species. The solvent system preferably is a good solvent for the polymer (or polymers) within the coating layer and for the therapeutic agent. The particular solvent species that make up the solvent system may also be selected based on other characteristics, including drying rate and surface tension.

Preferred solvent-based techniques include, but are not limited to, spin coating techniques, web coating techniques, solvent spraying techniques, dipping techniques, techniques involving coating via mechanical suspension including air suspension, ink jet techniques, electrostatic techniques, and combinations of these processes. Typically, the therapeutic agent is dissolved or dispersed within a solvent, and the resulting solution contacted with a previously formed coating layer using, for example, one or more of these application techniques.

In some embodiments, barrier layers are formed over a therapeutic-agent-containing coating layer. For example, solvent-based techniques such as those discussed above can be used in which a polymer (or polymers) that comprises the barrier region is (are) first dissolved or dispersed in a solvent, and the resulting mixture is subsequently used to form the barrier layer. In other embodiments, the barrier layer is applied over the therapeutic-agent-containing coating layer using thermoplastic processing techniques. The barrier layer serves, for example, as a boundary layer to retard diffusion of the therapeutic agent, for instance, acting to prevent a burst phenomenon whereby much of the therapeutic agent is released immediately upon exposure of the device or a portion of the device to the implant or insertion site.

A wide variety of medical devices are formed in conjunction with the present invention. Examples of medical devices include implantable or insertable medical devices, for example, catheters (e.g., renal or vascular catheters such as balloon catheters), guide wires, balloons, filters (e.g., vena cava filters), stents (including coronary vascular stents, cerebral, urethral, ureteral, biliary, tracheal, gastrointestinal and esophageal stents), stent grafts, cerebral aneurysm filter coils (including Guglilmi detachable coils and metal coils), vascular grafts, myocardial plugs, patches, pacemakers and pacemaker leads, heart valves, biopsy devices, and any coated substrate (which can comprise, for example, glass, metal, polymer, ceramic and combinations thereof) that is implanted or inserted into the body and from which therapeutic agent is released. Examples of medical devices further include patches for delivery of therapeutic agent to intact skin and broken skin (including wounds); sutures, suture anchors, anastomosis clips and rings, tissue staples and ligating clips at surgical sites; orthopedic fixation devices such as interference screws in the ankle, knee, and hand areas, tacks for ligament attachment and meniscal repair, rods and pins for fracture fixation, screws and plates for craniomaxillofacial repair; dental devices such as void fillers following tooth extraction and guided-tissue-regeneration membrane films following periodontal surgery; and tissue engineering scaffolds for cartilage, bone, skin and other in vivo tissue regeneration.

The medical devices of the present invention include medical devices that are used for either systemic treatment or for the localized treatment of any mammalian tissue or organ. Non-limiting examples are tumors; organs including the heart, coronary and peripheral vascular system (referred to overall as “the vasculature”), lungs, trachea, esophagus, brain, liver, kidney, bladder, urethra and ureters, eye, intestines, stomach, pancreas, vagina, uterus, ovary, and prostate; skeletal muscle; smooth muscle; breast; dermal tissue; cartilage; and bone.

Specific examples of medical devices for use in conjunction with the present invention include vascular stents, which deliver therapeutic agent into the vasculature for the treatment of restenosis. As used herein, “treatment” refers to the prevention of a disease or condition, the reduction or elimination of symptoms associated with a disease or condition, or the substantial or complete elimination of a disease or condition. Preferred subjects are mammalian subjects and more preferably human subjects.

“Therapeutic agents,” “pharmaceutically active agents,” “pharmaceutically active materials,” “drugs,” and other related terms may be used interchangeably herein and include genetic therapeutic agents, non-genetic therapeutic agents and cells. Therapeutic agents may be used singly or in combination. Therapeutic agents may be used singly or in combination. Therapeutic agents may be, for example, nonionic or they may be anionic and/or cationic in nature.

Exemplary non-genetic therapeutic agents for use in connection with the present invention include: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promoters; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, aminoglycosides and nitrofurantoin; (m) cytotoxic agents, cytostatic agents and cell proliferation affectors; (n) vasodilating agents; (O) agents that interfere with endogenous vasoactive mechanisms; (p) inhibitors of leukocyte recruitment, such as monoclonal antibodies; (q) cytokines; (r) hormones; and (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin.

Preferred non-genetic therapeutic agents include paclitaxel, sirolimus, everolimus, tacrolimus, Epo D, dexamethasone, estradiol, halofuginone, cilostazole, geldanamycin, ABT-578 (Abbott Laboratories), trapidil, liprostin, Actinomycin D, Resten-NG, Ap-17, abciximab, clopidogrel and Ridogrel, among others.

Exemplary genetic therapeutic agents for use in connection with the present invention include anti-sense DNA and RNA as well as DNA coding for the various proteins (as well as the proteins themselves): (a) anti-sense RNA, (b) tRNA or rRNA to replace defective or deficient endogenous molecules, (c) angiogenic and other factors including growth factors such as acidic and basic fibroblast growth factors, vascular endothelial growth factor, endothelial mitogenic growth factors, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers such as polyvinylpyrrolidone (PVP), SP1017 (SUPRATEK), lipids such as cationic lipids, liposomes, lipoplexes, nanoparticles, or microparticles, with and without targeting sequences such as the protein transduction domain (PTD).

Cells for use in connection with the present invention include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, skeletal myocytes or macrophage, or from an animal, bacterial or fungal source (xenogeneic), which can be genetically engineered, if desired, to deliver proteins of interest.

Numerous therapeutic agents, not necessarily exclusive of those listed above, have been identified as candidates for vascular treatment regimens, for example, as agents targeting restenosis. Such agents are useful for the practice of the present invention and include one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) ACE inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (O) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), rapamycin, cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Numerous additional therapeutic agents useful for the practice of the present invention are also disclosed in U.S. Pat. No. 5,733,925 assigned to NeoRx Corporation, the entire disclosure of which is incorporated by reference.

Therapeutic agents also include ablation agents, sufficient amounts of which will result in necrosis (death) of undesirable tissue, such as malignant tissue, prostatic tissue, and so forth. Examples include osmotic-stress-generating agents, for example, salts such as sodium chloride or potassium chloride; organic solvents, particularly those such as ethanol, which are toxic in high concentrations, while being well tolerated at lower concentrations; free-radical generating agents, for example, hydrogen peroxide, potassium peroxide or other agents that can form free radicals in tissue; basic agents such as sodium hydroxide; acidic agents such as acetic acid and formic acid; enzymes such as collagenase, hyaluronidase, pronase, and papain; oxidizing agents, such as sodium hypochlorite, hydrogen peroxide or potassium peroxide; tissue fixing agents, such as formaldehyde, acetaldehyde or glutaraldehyde; and naturally occurring coagulants, such as gengpin.

A wide range of therapeutic agent loadings can be used in connection with the dosage forms of the present invention, with the pharmaceutically effective amount being readily determined by those of ordinary skill in the art and ultimately depending, for example, upon the condition to be treated, the nature of the therapeutic agent itself, the tissue into which the dosage form is introduced, and so forth.

As will be appreciated by those of ordinary skill in the art, the release profile associated with the coating layer can be modified, for example, by altering the chemical composition, molecular weight, architecture, and so forth, of the polymer or polymers forming the therapeutic-agent-containing coating layer and/or by providing a barrier layer over the therapeutic-agent-containing coating layer.

Hence, in certain embodiments of the present invention, the drug release rate of the therapeutic agent is controlled by changing the hydrophilic/hydrophobic ratio of the polymeric constituents of the coating layer, such that the overall hydrophilicity of the coating layer is increased or decreased (or, viewed conversely, the overall hydrophobicity is increased or decreased). As will be appreciated by those of ordinary skill in the art, the ratio may be changed in a number of ways. For example, in some embodiments, the hydrophilicity of the coating layer can be increased by forming polymers using one or more hydrophilic monomers, such as hydroxyethylmethacrylate or other hydrophilic monomers including those numerous examples of hydrophilic monomers listed above for preparation of low and high T_{g} polymer blocks. In alternative embodiments, the hydrophobicity of the resulting copolymer is increased by forming copolymers with one or more hydrophobic monomers. Any one or more of a number of hydrophobic monomers can be used, such as methylmethacrylate or other hydrophobic monomers, including those numerous examples of hydrophobic monomers listed above for preparation of low and high T_{g} polymer blocks. Where the coating layer comprises a copolymer of hydrophilic and hydrophobic units, the ratio of hydrophilic units within the copolymer relative to hydrophobic units can be varied.

In other embodiments, release is modulated by including one or more biodisintegrable polymeric constituents in the coating layer, for example, a biodisintegrable polymer block. A “biodisintegrable polymer block” is a polymer block that undergoes dissolution, degradation, resorption and/or other disintegration process upon administration to a patient. Examples of biodisintegrable polymer blocks include the following: (a) polyester blocks, for example, polymers and copolymers of hydroxyacids and lactones, such as glycolic acid, lactic acid, tartronic acid, fumaric acid, hydroxybutyric acid, hydroxyvaleric acid, dioxanone, caprolactone and valerolactone, (b) polyanhydrides, for example, polymers and copolymers of various diacids such as sebacic acid and 1,6-bis(p-carboxyphoxy) alkanes, for instance, 1,6-bis(p-carboxyphoxy) hexane and 1,6-bis(p-carboxyphoxy) propane; (c) tyrosine-derived polycarbonates, and (d) polyorthoesters. Specific examples of biodisintegrable polymer blocks include polyester blocks such as poly(glycolic acid) blocks, poly(lactic acid) blocks, poly(lactic acid-co-glycolic acid) blocks, and polycaprolactone blocks.

### EXAMPLE

### Polystyrene-Coated Stainless Steel Stent

A stainless steel stent having a polystyrene coating is produced by the electrografting of chlorinated poly(ethyl acrylate) onto a stainless steel stent surface, followed by ATRP with styrene monomer. A 2-chloropropionate ethyl acrylate (cPEA) initiator is synthesized by reaction of 2-hydroxyethyl acrylate with 2-chloroproopionyl chloride in the presence of triethylamine to form (cPEA). The cPEA is dried over molecular sieves before electropolymerization, and the ethyl acrylate monomer is dried over calcium hydride and distilled under reduced pressure. N,N-Dimethylformamide (DMF) is dried over P₂O₅ and distilled under reduced pressure. Tetraethylammonium perchlorate (TEAP) is heated in vacuo at 80° C. for 12 hours, prior to use. Styrene (Aldrich) is dried over CaH₂ and distilled before use. Phenylethyl bromide (PEBr) (Aldrich) and HMTETA (Aldrich) are diluted in dried toluene. The Grubbs catalyst (Aldrich) and NiBr₂(PPh₃)₂ (Aldrich) are used as received and CuCl (Aldrich) and CuCl₂ (Aldrich) are purified by recrystallization in acetic acid, before use.

1. Synthesis of cPEA

cPEA is prepared by reaction of 10 mL of 2-chloropropionyl chloride (0.1 mol dissolved in 20 mL of tetrahydrofuran (THF)) with 5.74 mL of 2-hydroxyethyl acrylate (0.05 mol) and 6.97 mL of triethylamine (0.05 mol) dissolved in 60 mL of dried THF. Then, 20 hours later, the triethylamine hydrochloride byproduct is filtered out and washed with THF. Upon THF evaporation from the filtrate, a liquid residue is left, which is extracted by CHCl₃, washed three times with water, eluted through a silica gel column by CHCl₃, and dried over MgSO₄.

After distillation, cPEA is recovered.

2. Electrografted Stent

Stainless steel stents are electrografted with poly(cPEA) and poly(cPEA-co-EA) from a DMF solution containing TEAP (0.05 M) and either cPEA (0.5 M) or a mixture of cPEA (0.5 M) and EA (0.5M) by scanning the potential up to the maximum of the first peak and holding this potential until the current decreases dramatically. Complete cathodic passivation may require two or more scans. The substrate is then washed with pure DMF and acetonitrile and dried.

3. Polymerization of Styrene via ATRP

The styrene polymerization is performed in closed tubes under nitrogen. In a tube is added 68 mg (0.082 mmol for a targeted DP=950) of the Grubbs catalyst, together with the electrografted stainless steel stent. The tube is closed, evacuated and filled with nitrogen. Then, 2 mL of dry toluene, 9 mL of styrene (78.5 mmol) and 0.23 mL of PEBr (0.082 mmol; targeted DP=950) are then added through a rubber septum making the steel stent completely immersed. The tube is placed on a magnetic stirrer and heated at 100° C. for 18 hours. After polymerization, the steel stent is extensively washed with THF, and the polymer formed in solution is recovered by precipitation in methanol.

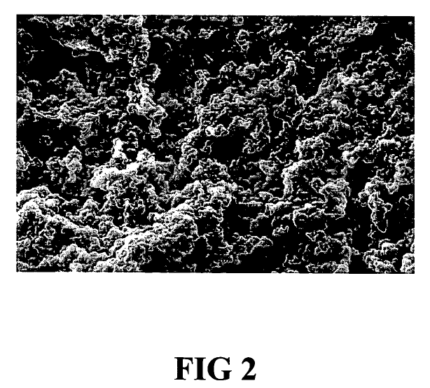

A representative surface of a typical polystyrene-grafted stainless steel stent produced according to the methods described above is shown in FIG. 2, which is a scanning electron micrograph of a polystyrene-coated steel plate, published in Claes et al., Polymer Coating of Steel by a Combination of Electrografting and Atom-Transfer Radical Polymerization, Macromolecules, Web release No. 0217130 (Jul. 19, 2003).

Although various embodiments are specifically illustrated and described herein, it will be appreciated that modifications and variations of the present invention are covered by the above teachings and are within the purview of the appended claims without departing from the spirit and intended scope of the invention.

What is claimed is:

1. A medical device comprising an electrically conductive substrate and a coating layer that covers at least a portion of said electrically conductive substrate, wherein said coating layer comprises a polymer that is made by a process comprising (a) electrochemically linking a free radical polymerization initiator to a surface of said electrically conductive substrate, and (b) conducting a free radical polymerization reaction in the presence of one or more free radical polymerizable monomers.

2. The device of claim 1, wherein said electrically conductive substrate comprises an elemental transition metal or alloy selected from titanium, platinum, stainless steel, nickel-titanium alloy, gold, cobalt-chromium alloy, and platinum-enriched stainless steel.

3. The device of claim 1, wherein said initiator is electrochemically linked to said electrically conductive substrate surface by applying a cathodic electric potential to said electrically conductive substrate in the presence of said initiator.

4. The device of claim 1, wherein said free radical polymerization reaction is an atom-transfer radical polymerization reaction.

5. The device of claim 1, wherein said initiator comprises an electrochemically linkable group selected from alkyl halides with one or more activating groups on an a carbon of the alkyl halide.

6. The device of claim 1, wherein said initiator is a polyhalogenated compound comprising an electrochemically linkable group selected from an —N—X group, an —S—X group, and an —O—X group, wherein X is a halogen atom.

7. The device of claim 1, wherein said initiator comprises an acrylate group and a 2-chloropropionate group.

8. The device of claim 1, wherein the initiator comprises a copolymer of ethyl acrylate and 2-chloropropionate.

9. The device of claim 1, wherein said electrochemically linked free radical polymerization initiator is a free radical terminated polymer.

10. The device of claim 1, wherein said one or more free radical polymerizable monomers are selected from alkyl acrylate monomers, hydroxyalkyl acrylate monomers, alkyl methacrylate monomer, hydroxyalkyl methacrylate monomers, acrylonitrile monomer, methacrylonitrile monomer, a vinyl ester monomers, styrene monomer, and substituted styrene monomers.

11. The device of claim 1, wherein said free radical polymerizable monomer is an unsaturated monomer.

12. The device of claim 1, wherein said one or more free radical polymerizable monomers comprise a macro-monomer having a free radical polymerizable group.

13. The device of claim 12, wherein the macro-monomer comprises a free radical polymerizable group and one or more of the following blocks: a polysiloxane block, a polyalkene block, a poly(halogenated alkene) block, a polyester block, a poly(vinyl aromatic) block, a polyethylene oxide block, a polyvinylpyrrolidone block, a polyacrylate block, a polymethacrylate block, or a poly(vinyl ether) block.

14. The device of claim 1, wherein said polymer comprises (i) a low T_{g} polymer block and (ii) a high T_{g} polymer block,

15. The device of claim 14, wherein said initiator comprises a free radical terminated low T_{g} polymer block.

16. The device of claim 14, wherein said low T_{g} block is selected from a polyacrylate block, a polymethacrylate block, a poly(vinyl ether) block, a polyester block, a polyalkene block, a poly(halogenated alkene) block, and a poly(siloxane) block.

17. The device of claim 14, wherein said high T_{g} block is selected from a poly(vinyl aromatic) block and a polyalkyl(meth)acrylate block.

18. The device of claim 1, wherein said polymer comprises a low T_{g} block and a graft copolymer block comprising a main chain and a plurality of side chains, and wherein the radical polymerization reaction is conducted in the presence of a macro-monomer comprising said side chain and a free radical polymerizable end group.

19. The device of claim 18, wherein said graft copolymer block comprises a biodisintegrable chain or a high T_{g} polymer chain.

20. The device of claim 1, wherein said coating layer further comprises a therapeutic agent.

21. The device of claim 20, wherein said therapeutic agent is selected from one or more of the group consisting of anti-thrombotic agents, anti-proliferative agents, anti-inflammatory agents, anti-migratory agents, agents affecting extracellular matrix production and organization, antineoplastic agents, anti-mitotic agents, anesthetic agents, anti-coagulants, vascular cell growth promoters, vascular cell growth inhibitors, cholesterol-lowering agents, vasodilating agents, and agents that interfere with endogenous vasoactive mechanisms.

22. The device of claim 20, wherein a therapeutic agent is introduced into the coating layer by (a) providing a solution comprising (i) a solvent system and (ii) said therapeutic agent; and (b) contacting said solution with said coating layer.

23. The device of claim 1, wherein said medical device is an implantable or insertable medical device.

24. The device of claim 23, wherein said implantable or insertable medical device is selected from a catheter, a guide wire, a balloon, a filter, a stent, a stent graft, a vascular graft, a vascular patch and a shunt.

25. The device of claim 23, wherein said implantable or insertable medical device is adapted for implantation or insertion into the coronary vasculature, peripheral vascular system, esophagus, trachea, colon, biliary tract, urinary tract, prostate or brain.
